(19) United States
(12) Patent Application Publication
(10) Pub. No.: US 2026/0167589 A1
(43) Pub. Date: Jun. 18, 2026

(54) METHODS AND SYSTEMS FOR IMPROVING ETHYLENE YIELD FROM ISO-PENTANE FEED BY REVERSE ISOMERIZATION AND SEPARATION

(71) Applicant: SABIC Global Technologies B.V., Bergen OP Zoom (NL)

(72) Inventors: Debdut S. Roy, Bangalore (IN); Prashil Prakash Lakhete, Bangalore (IN); Hareesh Shamrao Deshpande, Bangalore (IN)

(21) Appl. No.: 19/124,929

(22) PCT Filed: Oct. 27, 2023
(86) PCT No.: PCT/IB2023/060860

§ 371 Date: Apr. 28, 2025

(30) Foreign Application Priority Data
Oct. 28, 2022 (EP) 22204392.9

(51) Int. Cl. C07C 4/04; B01D 3/14; B01D 9/00; B01J 19/24

(52) U.S. Cl. CPC C07C 4/04; B01D 3/143; B01D 9/0059; B01J 19/245

(57) ABSTRACT

Systems and methods for the higher yield production of ethylene from pentane hydrocarbon streams containing iso-pentane are provided. The methods may include supplying a pentane hydrocarbon stream containing iso-pentane to a first reverse isomerization unit to produce a first isomerized pentane stream containing n-pentane and iso-pentane. The method may further include separating, at a first separation unit, n-pentane from the first isomerized pentane stream to generate a first n-pentane stream and an iso-pentane rich stream. The method may also include supplying the iso-pentane rich stream to a second reverse isomerization unit to produce a second isomerized pentane stream containing n-pentane and iso-pentane and combining the second isomerized pentane stream and the first n-pentane stream to produce a n-pentane rich stream. The method may further include supplying the n-pentane rich stream to a steam cracker to produce a cracked product containing ethylene and propylene.

### TECHNICAL FIELD

The present disclosure generally relates to systems and methods for increasing ethylene production from iso-pentane feeds. More specifically, the present disclosure relates to systems and methods for isomerizing a hydrocarbon feed containing iso-pentane, separating n-pentane from the isomerized hydrocarbon feed, and isomerizing the hydrocarbon feed following n-pentane separation before feeding the isomerized hydrocarbon feed and the separated n-pentane to a steam cracker, resulting in greater ethylene yield.

### BACKGROUND

Ethylene is a desirable industrial compound having a worldwide production greater than that of any other organic compound. Accordingly, ethylene is widely used in the chemical industry, particularly as a feedstock for the production of polyethylene. In addition to other production methods, ethylene may be generated by the cracking of pentane streams obtained from natural gas liquids (NGLs) at a steam cracker. However, poor yields of ethylene are often obtained from the cracking of pentane streams due to the presence of iso-pentane (iso-C5) isomer which is relatively non-reactive in the steam cracker. Accordingly, methods and systems capable of increasing the efficiency of ethylene production from pentane streams, particularly iso-pentane streams, is desirable.

### SUMMARY

To address these shortcomings in the art, Applicant has developed systems and methods for isomerizing a hydrocarbon feed containing iso-pentane, separating n-pentane from the isomerized hydrocarbon feed, and isomerizing the hydrocarbon feed following n-pentane separation before feeding the isomerized hydrocarbon feed and the separated n-pentane to a steam cracker, resulting in greater ethylene yield, according to the exemplary embodiments disclosed herein.

Systems and methods for the higher yield production of ethylene from pentane hydrocarbon streams containing iso-pentane are provided. In certain embodiments, the method for production of ethylene from a pentane hydrocarbon stream may include: supplying a pentane hydrocarbon stream containing iso-pentane to a first reverse isomerization unit to produce a first isomerized pentane stream, the first isomerized pentane stream containing n-pentane and iso-pentane; separating, at a first separation unit, n-pentane from the first isomerized pentane stream to generate a first n-pentane stream and an iso-pentane rich stream; supplying the iso-pentane rich stream to a second reverse isomerization unit to produce a second isomerized pentane stream containing n-pentane and iso-pentane; combining the second isomerized pentane stream and the first n-pentane stream to produce a n-pentane rich stream; and supplying the n-pentane rich stream to a steam cracker to produce a cracked product containing ethylene and propylene.

In some embodiments of the method, the yield of ethylene in the cracked product is greater than about 22 wt. % of the cracked product when the pentane hydrocarbon stream contains 100 mol % iso-pentane. In some embodiments of the method, the n-pentane rich stream contains more n-pentane than iso-pentane or neo-pentane. In some embodiments of the method, the n-pentane rich stream contains at least 45 mol % n-pentane. In at least some embodiments of the method, the pentane hydrocarbon stream is an iso-pentane stream derived from natural gas liquids. In at least some embodiments of the method, the pentane hydrocarbon stream is a mixture of an iso-pentane stream derived from natural gas liquids (NGLs) and a pentane stream derived from the cracking of the n-pentane rich stream to produce ethylene.

In some embodiments, the method further includes treating, at a hydrotreater unit, the pentane hydrocarbon stream to remove impurities prior to supplying the pentane hydrocarbon stream to the first reverse isomerization unit. In some embodiments, the method further includes: cracking the n-pentane rich stream to produce a mixed ethylene stream containing ethylene, one or more additional cracked products, n-pentane and iso-pentane; separating, at one or more cracker separation units, the mixed ethylene stream to produce an ethylene stream and a recycled pentane stream containing n-pentane and iso-pentane; and combining the recycled pentane stream and an iso-pentane stream derived from NGLs to form the pentane hydrocarbon stream.

In some embodiments, the method further includes hydrotreating the iso-pentane stream derived from NGLs at a hydrotreater to form a hydrotreated iso-pentane stream, and combining the hydrotreated iso-pentane stream with the recycled pentane stream before being fed to the first reverse isomerization unit. In at least some embodiments of the method, the second isomerized pentane stream contains n-pentane, iso-pentane, and neo-pentane, and prior to combining the second isomerized pentane stream and the n-pentane stream, the method further includes: separating, at a neo-pentane separation unit, neo-pentane from the second isomerized pentane stream to produce a neo-pentane stream and an enriched second isomerized pentane stream containing n-pentane and iso-pentane; combining the enriched second isomerized pentane stream and the first n-pentane stream to produce the n-pentane rich stream.

In at least some embodiments of the method, prior to combining the second isomerized pentane stream and the n-pentane stream, the method further includes: separating, at a second separation unit, n-pentane from the second isomerized pentane stream to generate a second n-pentane stream and a second iso-pentane rich stream; supplying the second iso-pentane rich stream to a third reverse isomerization unit to produce a third isomerized pentane stream containing n-pentane, iso-pentane, and neo-pentane; combining the third isomerized pentane stream and the first and second n-pentane streams to produce the n-pentane rich stream.

In some embodiments of the method, prior to combining the second isomerized pentane stream and the n-pentane stream, the method further includes: separating, at the second separation unit, n-pentane from the second isomerized pentane stream to generate the second n-pentane stream and the second iso-pentane rich stream; supplying the second iso-pentane rich stream to the third reverse isomerization unit to produce the third isomerized pentane stream containing n-pentane, iso-pentane, and neo-pentane; separating, at a neo-pentane separation unit, neo-pentane from the third isomerized pentane stream to produce a neo-pentane stream and an enriched third isomerized pentane stream containing n-pentane and iso-pentane; combining the enriched third isomerized pentane stream and the first and the second n-pentane streams to produce the n-pentane rich stream. In some embodiments, the method further includes feeding the neo-pentane stream to the first reverse isomerization unit.

The present disclosure also provides a method for the production of ethylene from a pentane hydrocarbon stream containing iso-pentane that includes: supplying a pentane hydrocarbon stream to a first reverse isomerization unit to produce a first isomerized pentane stream, the first isomerized pentane stream containing n-pentane and iso-pentane; supplying the first isomerized pentane stream to a first separation unit to separate n-pentane from the first isomerized pentane stream to form a first n-pentane stream and a first iso-pentane rich stream; supplying the first iso-pentane rich stream to one or more reverse isomerization and separation units until a combination of the separated n-pentane streams and a resultant iso-pentane rich stream forms a n-pentane rich stream containing greater than 40 mole percent of n-pentane; and supplying the n-pentane rich stream to a steam cracker to produce a cracked product containing ethylene and propylene.

In some embodiments, the method further includes supplying the first iso-pentane rich stream to a second reverse isomerization unit to produce a second isomerized pentane stream containing n-pentane and iso-pentane; combining the second isomerized pentane stream and the first n-pentane stream to produce the n-pentane rich stream. In some embodiments of the method, a yield of ethylene in the cracked product is greater than about 22 wt. % of the cracked product when the pentane hydrocarbon stream contains 100 mol. % iso-pentane. In some embodiments of the method, the n-pentane rich stream contains greater than 45 mole percent of n-pentane. In some embodiments of the method, the n-pentane rich stream contains greater than 50 mole percent of n-pentane.

The present disclosure also provides a system for the production of ethylene from a pentane hydrocarbon stream containing iso-pentane. In some embodiments, the system includes: a first reverse isomerization unit operable to receive a pentane stream and allow for isomerization of the pentane stream to produce a first isomerized pentane stream containing n-pentane and iso-pentane; a first separation unit operable to receive the first isomerized pentane stream and allow for separation of n-pentane from the isomerized pentane stream to form a first n-pentane stream and an iso-pentane rich stream; a second reverse isomerization unit operable to receive the iso-pentane rich stream and allow for isomerization of the iso-pentane rich stream to produce a second isomerized pentane stream containing n-pentane and iso-pentane; and a steam cracker operable to receive the second isomerized pentane stream and the first n-pentane stream as a n-pentane rich stream, the steam cracker further operable to crack the n-pentane rich stream to produce a cracked product containing ethylene and propylene.

In some embodiments, the system further includes: a hydrotreater unit operable to receive the pentane hydrocarbon stream and remove impurities from the pentane hydrocarbon stream to form a purified pentane stream, the hydrotreater unit further operable to feed the purified pentane stream to the first reverse isomerization unit. In some embodiments, the system further includes: one or more cracker units to crack the n-pentane rich stream to produce a mixed ethylene stream containing ethylene, pentane, and one or more additional cracked products; one or more cracker separation units operable to receive the mixed ethylene stream from the steam cracker and to produce an ethylene stream and a recycled pentane stream; and the first reverse isomerization unit is operable to receive a combined pentane stream containing the recycled pentane stream and the pentane stream or the purified pentane stream.

In some embodiments of the system, the second isomerized pentane stream contains n-pentane, iso-pentane, and neo-pentane, and the system further includes: a neo-pentane separation unit operable to receive the second isomerized pentane stream and separate neo-pentane from the second isomerized pentane stream to produce a neo-pentane stream and an enriched second isomerized pentane stream containing n-pentane and iso-pentane; and the steam cracker operable to receive the combined n-pentane rich stream containing the combination of the first n-pentane stream and the enriched second isomerized pentane stream, the steam cracker further operable to crack the n-pentane rich stream to produce ethylene and one or more additional cracked products.

In some embodiments, the system further includes: a second separation unit operable to receive the second isomerized pentane stream and separate n-pentane from the second isomerized pentane stream to generate a second n-pentane stream and a second iso-pentane rich stream; a third reverse isomerization unit operable to receive the second iso-pentane rich stream and isomerize the second iso-pentane rich stream to produce a third isomerized pentane stream containing n-pentane, iso-pentane, and neo-pentane; wherein the steam cracker is operable to receive a combined n-pentane rich stream containing the combination of the first n-pentane stream, the second n-pentane stream, and the third isomerized pentane stream, the steam cracker further operable to crack the n-pentane rich stream to produce ethylene and one or more additional cracked products.

In some embodiments of the system, the second separation unit is operable to receive the second isomerized pentane stream and separate n-pentane from the second isomerized pentane stream to generate a second n-pentane stream and a second iso-pentane rich stream; the third reverse isomerization unit operable to receive the second iso-pentane rich stream and isomerize the second iso-pentane rich stream to produce a third isomerized pentane stream containing n-pentane, iso-pentane, and neo-pentane; the neo-pentane separation unit operable to receive the third isomerized pentane stream and to separate neo-pentane from the third isomerized pentane stream to produce a neo-pentane stream and an enriched third isomerized pentane stream containing n-pentane and iso-pentane; and the steam cracker is operable to receive a combined n-pentane rich stream containing the combination of one or more n-pentane streams and the enriched third isomerized pentane stream, the steam cracker further operable to crack the n-pentane rich stream to produce ethylene and one or more additional cracked products.

In some embodiments of the system, the neo-pentane separation unit is coupled with the first reverse isomerization unit, and the neo-pentane separation unit operable to recycle the neo-pentane stream to the first reverse isomerization unit. In some embodiments of the system, the first separation unit and the second separation unit are one or more of a distillation column, a crystallization unit, and any combination thereof. In some embodiments of the system, at least one of the first reverse isomerization unit, the second reverse isomerization unit, and the third reverse isomerization unit is configured to operate in the presence of a platinum or a palladium catalyst supported on a solid matrix. In some embodiments of the system, the solid matrix is a chlorinated alumina, a zeolite matrix, a mordenite matrix, a sulfated zirconia, or any combination thereof. In some embodiments of the system, at least one of the first reverse isomerization unit, the second reverse isomerization unit, and the third reverse isomerization unit is configured to operate at a temperature of from about 150° C. to about 450° C. in the presence of hydrogen. The hydrogen generally needs to be removed using known unit operations prior to sending the product stream containing iso-C5, n-C5, and neo-C5 to the steam cracker.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to illustrate embodiments of the disclosure more clearly.

FIG. 1 is a graphical representation of a system and method for producing ethylene from an iso-pentane hydrocarbon feed that includes a single reverse isomerization unit and a steam cracker, according to an exemplary embodiment of the present disclosure.

FIG. 2 is a graphical representation of a system and method for producing ethylene from an iso-pentane hydrocarbon feed that includes two reverse isomerization units and a separation unit, according to an exemplary embodiment of the present disclosure.

FIG. 3 is a graphical representation of a system and method for producing ethylene from an iso-pentane hydrocarbon feed that includes three reverse isomerization units and two separation units, according to an exemplary embodiment of the present disclosure.

FIG. 4 is a graphical representation of a system and method for producing ethylene from an iso-pentane hydrocarbon feed that includes three reverse isomerization units, two separation units, and a neo-pentane separation unit, according to an exemplary embodiment of the present disclosure.

FIG. 5 is a graphical representation of the steam cracker input stream and ethylene yield for a system and method having a single reverse isomerization unit as described in Example 1, according to an exemplary embodiment of the present disclosure.

FIG. 6 is a graphical representation of the steam cracker input stream and ethylene yield for a system and method having two reverse isomerization units and a separation unit as described in Example 2, according to an exemplary embodiment of the present disclosure.

FIG. 7 is a graphical representation of the steam cracker input stream and ethylene yield for a system and method having three reverse isomerization units and two separation units as described in Example 3, according to an exemplary embodiment of the present disclosure.

### DETAILED DESCRIPTION

The present disclosure describes various embodiments related to processes, methods, and systems for isomerizing a hydrocarbon feed containing iso-pentane, separating n-pentane from the isomerized hydrocarbon feed, and isomerizing the hydrocarbon feed following n-pentane separation before feeding the isomerized hydrocarbon feed and the separated n-pentane to a steam cracker, resulting in greater ethylene yield. Further embodiments may be described and disclosed.

In the following description, numerous details are set forth in order to provide a thorough understanding of the various embodiments. In other instances, well-known processes, devices, and systems may not have been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Disclosed here are systems and methods for increasing ethylene production from hydrocarbon feed streams containing iso-pentane. The iso-pentane isomer yields very low cracking products and thus is generally recycled along with fresh iso-pentane to the isomerization unit prior to being fed into the stream cracker. However, the Applicant has determined that iso-pentane isomerization to n-pentane is an equilibrium-controlled reaction and that isomerized iso-pentane streams contain no more than about 40 mol. % n-pentane following a single pass through a reverse isomerization reactor. The presently disclosed systems and methods are operable to improve ethylene yield from hydrocarbon streams containing iso-pentane by providing greater relative amount of n-pentane isomer to the steam cracker feed, thereby reducing the less-reactive iso-pentane load to the stream cracker and reducing the recycle load of iso-pentane. In particular, the present disclosure provides systems and methods for isomerizing a hydrocarbon feed containing iso-pentane, separating n-pentane from the isomerized hydrocarbon feed, and isomerizing the hydrocarbon feed following n-pentane separation before feeding the isomerized hydrocarbon feed and the separated n-pentane to a steam cracker, resulting in greater ethylene yield.

FIG. 1 is a graphical representation of an exemplary system 100 and method for producing ethylene and other cracked products 160 from a pentane hydrocarbon feed stream 115 containing iso-pentane 110 that includes a single reverse isomerization unit 130 and a steam cracker 140, according to an exemplary embodiment of the present disclosure. As depicted in FIG. 1, the pentane hydrocarbon feed stream 115 containing iso-pentane 110 may be fed to a hydrotreater 120 operable to treat the hydrocarbon feed stream 115 to remove impurities prior to supplying the pentane hydrocarbon stream to reverse isomerization unit 130. In particular, hydrotreater 120 is operable to hydrotreat the hydrocarbon feed stream 115 to form a hydrotreated iso-pentane stream 125. Hydrotreater 120 is also fluidly coupled with reverse isomerization unit 130 such that hydrotreated iso-pentane stream 125 may be discharged from hydrotreater 120 and fed to the reverse isomerization unit 130.

Reverse isomerization unit 130 is operable to isomerize the hydrotreated iso-pentane stream 125 to produce an isomerized pentane stream 135. The isomerized pentane stream 135 may contain n-pentane (n-C5) and neo-pentane (neo-C5) in addition to iso-pentane (iso-C5). The reverse isomerization unit 130 is fluidly coupled to steam cracker 140 such that the isomerized pentane stream 135 produced and discharged by the reverse isomerization unit 130 may be fed to the steam cracker 140. Steam cracker 140 is operable to receive the isomerized pentane stream 135 and crack the isomerized pentane stream 135 to produce a cracked mixed ethylene stream 145 containing ethylene, one or more additional cracked products, and pentane. The steam cracker 140 is fluidly coupled with one or more cracker separation units 150 such that the cracked mixed ethylene stream 145 may be discharged from the steam cracker 140 and fed to the one or more cracker separation units 150.

The one or more cracker separation units 150 in system 100 may be operable to separate the cracked mixed ethylene stream 145 to produce an ethylene stream 155 and a recycled pentane stream 156. The ethylene stream 155 may contain ethylene as well as one or more other cracked products 160. The recycled pentane stream 156 may include n-pentane, iso-pentane, and neo-pentane. The one or more cracker separation units 150 may be fluidly coupled with the reverse isomerization unit 130 such that the recycled pentane stream 156 discharged from the one or more cracker separation units 150 may be fed or otherwise recycled to the reverse isomerization unit 130.

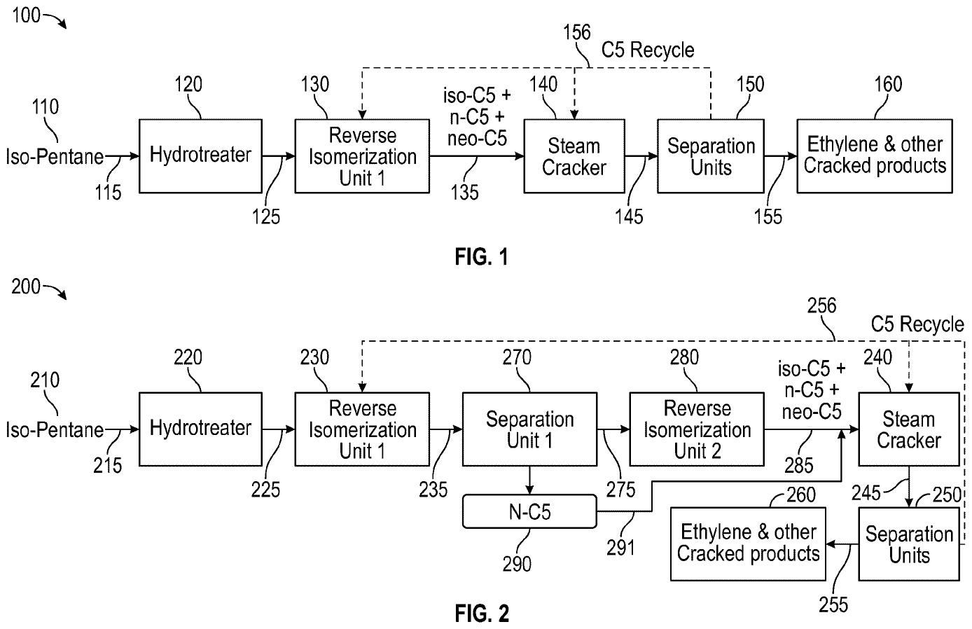

FIG. 2 is a graphical representation of an exemplary system 200 and method for generating improved ethylene yields from steam cracking a pentane hydrocarbon feed stream 215 containing iso-pentane 210 at a steam cracker 240, as compared to system 100 depicted in FIG. 1. As depicted in FIG. 2, the exemplary system 200 and method includes two reverse isomerization units 230, 280 and a separation unit 270. Before supplying the pentane hydrocarbon feed stream 215 containing iso-pentane 210 to the first reverse isomerization unit 230, the pentane hydrocarbon feed stream 215 containing iso-pentane 210 may optionally be fed to a hydrotreater 220 operable to treat the hydrocarbon feed stream 215 to remove impurities, such as nitrogen (N-), oxygen (O-), and sulfur (S-) containing impurities. In particular, optional hydrotreater 220 may be operable to hydrotreat the hydrocarbon feed stream 215 to form a hydrotreated iso-pentane stream 225. Optional hydrotreater 220 may also be fluidly coupled to the first reverse isomerization unit 230 such that the hydrotreated iso-pentane stream 225 may be discharged from hydrotreater 220 and fed to the first reverse isomerization unit 230.

The pentane hydrocarbon feed stream 215 containing iso-pentane 210 may be supplied to the first reverse isomerization unit 230 to produce a first isomerized pentane stream 235. The pentane hydrocarbon feed stream 215 containing iso-pentane 210 may be supplied directly to the first reverse isomerization unit 230 or may be supplied to the first reverse isomerization unit 230 in the form of hydrotreated iso-pentane stream 225 after being optionally treated at hydrotreater 220. The first reverse isomerization unit 230 is operable to isomerize the pentane hydrocarbon feed stream 215 containing iso-pentane 210 or the optionally hydrotreated iso-pentane stream 225 to produce a first isomerized pentane stream 235.

The first reverse isomerization unit 230, as well as second reverse isomerization unit 280 and any other reverse isomerization units that may be implemented in system 200, may operate using or containing a platinum (Pt) or palladium (Pd) containing catalyst supported on a solid matrix. The solid matrix may contain, for example, chlorinated alumina, zeolites, mordenite, sulfated zirconia. The reverse isomerization units 230, 280 may operate at a temperature of from about 150° C. to about 450° C. in the presence of hydrogen. Operation of reverse isomerization units 230, 280 causes the conversion of a portion of the iso-pentane isomers to n-pentane and neo-pentane monomers in a process that is generally equilibrium-limited. As a result, the first isomerized pentane stream 235 may contain n-pentane (n-C5) and neo-pentane (neo-C5) in addition to iso-pentane (iso-C5).

The first reverse isomerization unit 230 may be fluidly coupled to a first separation unit 270 configured to receive the first isomerized pentane stream 235 from the first reverse isomerization unit 230. The first separation unit 270 is operable to separate n-pentane from the first isomerized pentane stream 235 to generate a first n-pentane stream 290 and an iso-pentane rich stream 275. The first separation unit 270 can be, for example, a distillation column or a crystallizer unit, in addition to other forms of separation units operable to separate n-pentane from the first isomerized pentane stream 235 to generate a pentane stream enriched in iso-pentane, such as iso-pentane rich stream 275.

First separation unit 270 may be fluidly coupled to a second reverse isomerization unit 280 configured to receive the iso-pentane rich stream 275 from the first separation unit 270. The second reverse isomerization unit 280 is operable to produce a second isomerized pentane stream 285. The second isomerized pentane stream 285 may contain n-pentane in addition to iso-pentane. The second isomerized pentane stream 285 may also contain neo-pentane. The product stream 285 from the second reverse isomerization unit 280 may contain hydrogen and light gases along with C5 reactants and products. The hydrogen and light gases, mainly methane (CH4), generally needs to be removed using known unit operations before sending the product stream to steam cracker 240. The second reverse isomerization unit 280 may be fluidly coupled to a steam cracker 240 configured to receive the second isomerized pentane stream 285 from the second reverse isomerization unit 280. The first separation unit 270 may also be fluidly coupled to the steam cracker 240 such that the first n-pentane stream 290 may be supplied to the steam cracker 240 in the form of first n-pentane feed stream 291. The supplying of separated n-pentane 290 in the form of first n-pentane feed stream 291 to steam cracker 240 results in steam cracker 240 producing higher ethylene yields. The first n-pentane feed stream 291 may be supplied to steam cracker 240 directly or may be combined with the second isomerized pentane stream 285 prior to being supplied to steam cracker 240.

Steam cracker 240 is operable to receive the first n-pentane feed stream 291 and second isomerized pentane stream 285, either separately or in the form of a combined stream, and crack the first n-pentane feed stream 291 and second isomerized pentane stream 285 to produce a cracked mixed ethylene stream 245 containing ethylene, one or more additional cracked products, and pentane. The steam cracker 240 may be fluidly coupled with one or more cracker separation units 250 configured to receive the cracked mixed ethylene stream 245 from the steam cracker 240. In this manner, the cracked mixed ethylene stream 245 may be discharged from the steam cracker 240 and fed to the one or more cracker separation units 250 operable to separate the cracked mixed ethylene stream 245 to produce an ethylene stream 255 and a recycled pentane stream 256. The ethylene stream 255 may contain ethylene as well as one or more other cracked products 260. The recycled pentane stream 256 may include n-pentane, iso-pentane, and neo-pentane. The one or more cracker separation units 250 may be fluidly coupled with the first reverse isomerization unit 230 such that the recycled pentane stream 256 discharged from the one or more cracker separation units 250 may be fed or otherwise recycled to the first reverse isomerization unit 230.

The recycled pentane stream 256 may be supplied directly to the first reverse isomerization unit 230 or may be combined with the hydrotreated iso-pentane stream 225 or the pentane hydrocarbon feed stream 215 containing iso-pentane 210 prior to being fed to the first reverse isomerization unit 230. The first reverse isomerization unit 230 and the second reverse isomerization unit 280 may operate at the same conditions and with the same catalysts or may operate at different conditions and use different catalysts. The one or more cracker separation units 250 may also be fluidly coupled with the steam cracker 240 such that at least a portion of the recycled pentane stream 256 discharged from the one or more cracker separation units 250 may be fed or otherwise recycled to the steam cracker 240. In some instances, the one or more cracker separation units 250 may be configured to separate n-pentane from the cracked mixed ethylene stream 245 and feed the separate n-pentane directly back to the steam cracker 240 for conversion to products.

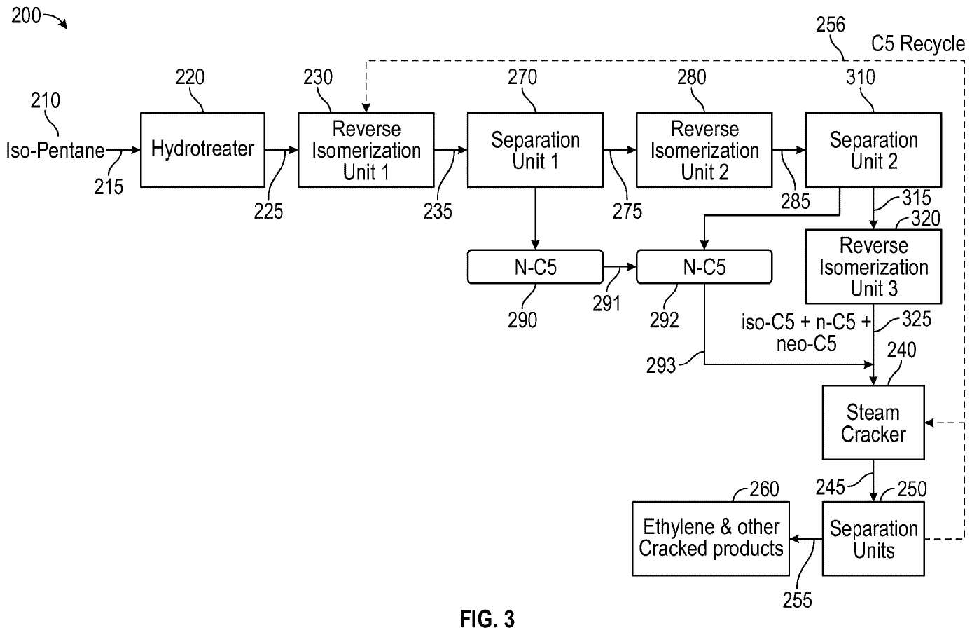

System 200 may include additional reverse isomerization units and separation units in order to further increase the n-pentane load to the steam cracker 240 resulting in even greater ethylene yields, as shown in FIG. 3. For example, as depicted in FIG. 3, the second reverse isomerization unit 280 may be fluidly coupled with a second separation unit 310 configured to receive the second isomerized pentane stream 285 from the second reverse isomerization unit 280. The second separation unit 310 may be operable to separate n-pentane from the second isomerized pentane stream 285 to generate a second n-pentane stream 292 and a second iso-pentane rich stream 315. As depicted in FIG. 3, the second separation unit 310 may be fluidly coupled to a third reverse isomerization unit 320 configured to receive the iso-pentane rich stream 315 from the second separation unit 310. The third reverse isomerization unit 320 is operable to produce a third isomerized pentane stream 325. The third isomerized pentane stream 325 may contain n-pentane in addition to iso-pentane. The third isomerized pentane stream 325 may also contain neo-pentane. The third reverse isomerization unit 320 may be fluidly coupled to the steam cracker 240 configured to receive the third isomerized pentane stream 325 from the third reverse isomerization unit 320.

The second separation unit 310 may also be fluidly coupled to the steam cracker 240 such that the second n-pentane stream 292 may be supplied to the steam cracker 240 in the form of n-pentane rich feed stream 293. The second n-pentane feed stream 292 may be supplied to steam cracker 240 directly or may be combined with the first n-pentane feed stream 291 and/or the third isomerized pentane stream 325 prior to being supplied to steam cracker 240. In some instances, such as that depicted in FIG. 3, the second n-pentane feed stream 292 may be combined with the first n-pentane feed stream 291 to form a combined n-pentane rich feed stream 293 and the combined n-pentane rich feed stream 293 fed or otherwise supplied to the steam cracker 240. Accordingly, steam cracker 240 is operable to receive the first n-pentane feed stream 291 and the second n-pentane feed stream, or combined n-pentane rich feed stream 293, as well as the third isomerized pentane stream 240, either separately or in the form of a combined stream, and crack the respective feed streams to produce a cracked mixed ethylene stream 245 containing ethylene, one or more additional cracked products, and pentane.

As described above with respect to FIG. 2, the steam cracker 240 may be fluidly coupled with one or more cracker separation units 250 configured to receive the cracked mixed ethylene stream 245 from the steam cracker 240 and separate the cracked mixed ethylene stream 245 to produce an ethylene stream 255 and the recycled pentane stream 256. The first separation unit 270 and the second separation unit 310 may or may not operate in the same manner or under the same conditions. For example, the first separation unit 270 may be a distillation unit and the second separation unit 310 may be a crystallization unit, or vice versa. Any number of forms of separation units and operating conditions are contemplated in the present disclosure so long as the separation units are operable to separate n-pentane from the hydrocarbon stream received at the one or more separation units.

In addition to the embodiments shown in FIG. 2 and FIG. 3, other embodiments are within the spirit and scope of the present disclosure. In particular, system 200 may include additional isomerization and separation units in order to convert even more of the iso-pentane in the input stream to n-pentane, thereby generating even greater ethylene yields at the steam cracker. However, the cost of implementing additional isomerization and separation units must be balanced with the resulting benefit of increased ethylene yield. Increasing the n-pentane load to steam cracker also reduces the recycle load of iso-pentane back to the first reverse isomerization unit, which in turn increases the efficiency of the system.

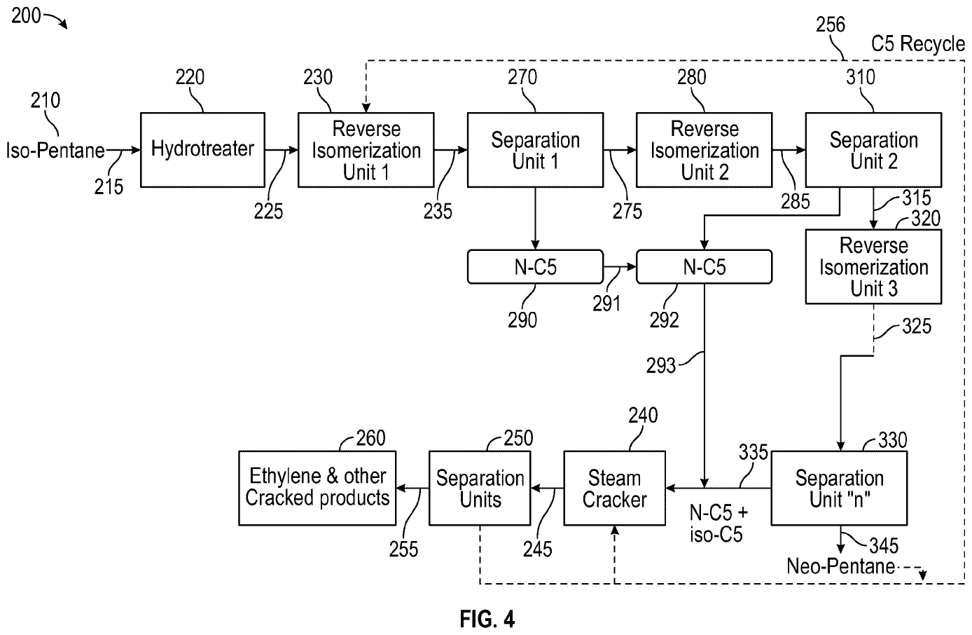

System 200 may also include one or more separation units placed in the flowpath between the last reverse isomerization unit and the steam cracker, as depicted for example in FIG. 4. As shown in FIG. 4, third reverse isomerization unit 320 is fluidly coupled with one or more terminal separation units 330 positioned between the terminal or last reverse isomerization unit in system 200 and the steam cracker 240. The one or more terminal separation units 330 may be configured to receive the final isomerized pentane stream, such as third isomerized pentane stream 325. The one or more terminal separation units 330 may be operable to separate one or more components from the final isomerized pentane stream (e.g., third isomerized pentane stream 325), in order to enrich the feed stream to the steam cracker 240 in n-pentane or otherwise remove less reactive iso-pentane, thereby increasing the efficiency of ethylene production at the steam cracker 240. In some instances, the one or more terminal separation units 330 may be operable to separate neo-pentane from the terminal isomerized pentane stream, such as third isomerized pentane stream 325, in order to produce a neo-pentane stream 345 and an enriched third isomerized pentane stream 335 containing n-pentane and iso-pentane. It is preferred to recycle neo-pentane back to the first reverse isomerization unit because equilibrium constraint selectivity causes the neo-pentane to convert to n-pentane. Therefore, neo-pentane stream 345 may be combined with recycled pentane stream 256 or otherwise supplied or fed to the first reverse isomerization unit 230, as shown in FIG. 4. The enriched third isomerized pentane stream 335 may be supplied or fed to steam cracker 240 either as an independent feed stream 335 or after combining the enriched third isomerized pentane stream 335 with the combined n-pentane rich feed stream 293. Separation of neo-pentane stream 345 and recycling the neo-pentane stream 345 to the first reverse isomerization unit 230 reduces the load on the steam cracker 240 thereby increasing the efficiency of steam cracker 240 operations resulting in improved ethylene production.

### EXAMPLES

The examples provided below illustrate selected aspects of the various methods and systems for the production of ethylene from a pentane hydrocarbon stream containing iso-pentane. While hydrogen is generally required for operation of the reverse isomerization unit(s) described herein, the hydrogen may be separated out of the product stream prior to sending the product stream to the steam cracker unit. All examples described here are on a hydrogen free basis as hydrogen is required only to enhance catalyst life and converting olefinic compounds, and does not affect equilibrium of C5 compounds.

### Example 1

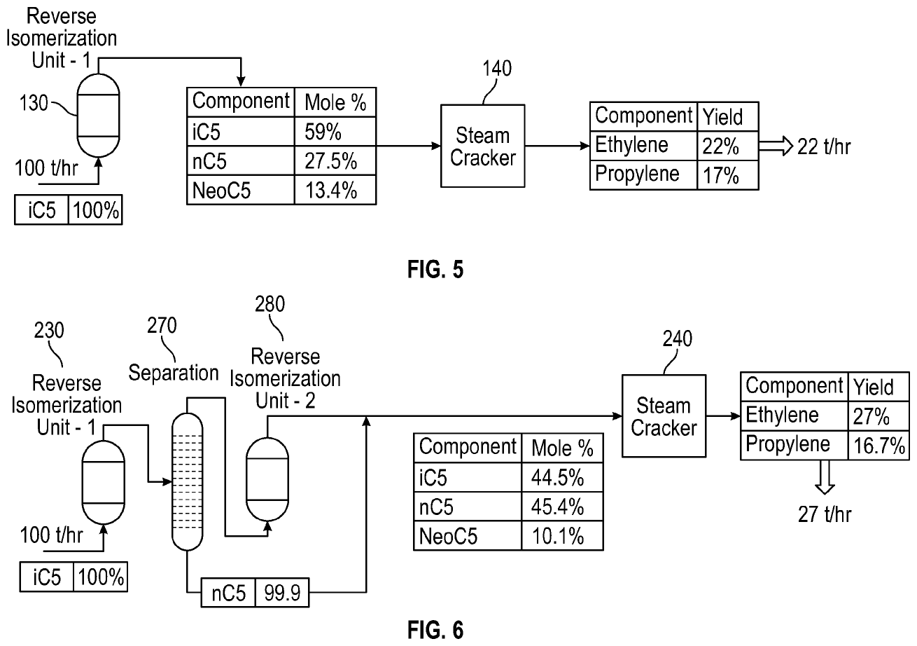

Ethylene yields for a pentane hydrocarbon stream containing 100 mol. % iso-pentane obtained by implementation of the exemplary embodiment of the system 100 and method provided in FIG. 1 is shown in FIG. 5. Ethylene yields and species distributions were obtained using Aspen Process simulator and Coilsim software. As shown in FIG. 5, if 100 ton/hour (t/hr) of a hydrocarbon stream containing 100 mol. % iso-pentane is fed to reverse isomerization unit 130, the equilibrium pentane composition of isomerized pentane stream 135 after reaction will be 59 mol. % iso-pentane, 27.5 mol. % n-pentane, and 13.4 mol. % neo-pentane. Feeding this isomerized pentane stream 135 to steam cracker 140 results in an ethylene yield around 22 wt. % and 17 wt. % propylene. Therefore, a 100 t/hr iso-pentane feed results in approximately 22 t/hr ethylene production using the system 100 shown in FIG. 1.

### Example 2

Ethylene yields for a pentane hydrocarbon stream containing 100 mol. % iso-pentane obtained by implementation of the exemplary embodiment of the system 200 and method provided in FIG. 2 is shown in FIG. 6. Ethylene yields and species distributions were obtained using Aspen Process simulator and Coilsim software. As shown in FIG. 6, if 100 t/hr of a hydrocarbon stream containing 100 mol. % iso-pentane is fed to first reverse isomerization unit 230 and the resulting equilibrium composition fed to first separation unit 270 to separate n-pentane from the unreacted iso-pentane and produced neo-pentane which is then fed to a second reverse isomerization unit 280 to produce an output stream combined with the separated n-pentane stream, the combined stream fed to the cracker 240 will be 44.5 mol. % iso-pentane, 45.4 mol. % n-pentane, and 10.1 mol. % neo-pentane. Feeding this pentane stream to steam cracker 240 results in an ethylene yield around 27 wt. % and 16.7 wt. % propylene. Therefore, a 100 t/hr iso-pentane feed results in approximately 27 t/hr ethylene production using the system 200 shown in FIG. 2. Therefore, implantation of system 200 shown in FIG. 2 results in an improvement of 4-5 t/hr in ethylene yield as compared to the system 100 shown in FIG. 1 described in Example 1.

### Example 3

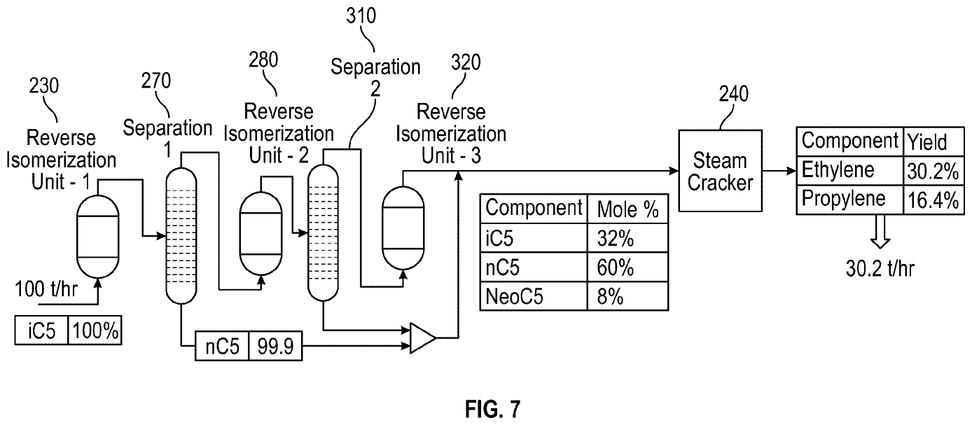

Ethylene yields for a pentane hydrocarbon stream containing 100 mol. % iso-pentane obtained by implementation of the exemplary embodiment of the system 200 and method provided in FIG. 3 is shown in FIG. 7. Ethylene yields and species distributions were obtained using Aspen Process simulator and Coilsim software. As shown in FIG. 7, if 100 t/hr of a hydrocarbon stream containing 100 mol. % iso-pentane is fed to first reverse isomerization unit 230, followed by separation at first separation unit 270, isomerization at second reverse isomerization unit 280, separation at second separation unit 310, and isomerization at third isomerization unit 320, the resulting combined pentane stream fed to the cracker 240 will contain 32 mol. % iso-pentane, 60 mol. % n-pentane, and 8 mol. % neo-pentane. Feeding this pentane stream to steam cracker 240 results in an ethylene yield around 30.2 wt. % and 16.4 wt. % propylene. Therefore, a 100 t/hr iso-pentane feed results in approximately 30 t/hr ethylene production using the system 200 shown in FIG. 3. Therefore, implementation of system 200 shown in FIG. 3 results in an improvement of 7-8 t/hr in ethylene yield as compared to the system 100 shown in FIG. 1 and described in Example 1. Implementation of system 200 shown in FIG. 3 also results in an improvement of approximately 3 t/hr in ethylene yield as compared to the system 200 shown in FIG. 2 and described in Example 2.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

1. A method for the production of ethylene from a pentane hydrocarbon stream containing iso-pentane, the method comprising:
supplying a pentane hydrocarbon stream containing iso-pentane to a first reverse isomerization unit to produce a first isomerized pentane stream, the first isomerized pentane stream containing n-pentane and iso-pentane;
separating, at a first separation unit, n-pentane from the first isomerized pentane stream to generate a first n-pentane stream and an iso-pentane rich stream;
supplying the iso-pentane rich stream to a second reverse isomerization unit to produce a second isomerized pentane stream containing n-pentane and iso-pentane;
combining the second isomerized pentane stream and the first n-pentane stream to produce a n-pentane rich stream; and
supplying the n-pentane rich stream to a steam cracker to produce a cracked product containing ethylene and propylene.

2. The method according to claim 1, wherein a yield of ethylene in the cracked product is greater than about 22 wt. % of the cracked product when the pentane hydrocarbon stream contains 100 mol. % iso-pentane.

3. The method according to claim 1, wherein the n-pentane rich stream contains more n-pentane than iso-pentane or neo-pentane.

4. The method according to claim 1, wherein the n-pentane rich stream contains at least 45 mol. % n-pentane.

5. The method according to claim 1, wherein the pentane hydrocarbon stream is an iso-pentane stream derived from natural gas liquids.

6. The method according to claim 1, wherein the pentane hydrocarbon stream is a mixture of an iso-pentane stream derived from natural gas liquids (NGLs) and a pentane stream derived from cracking of the n-pentane rich stream to produce ethylene.

7. The method according to claim 1, further comprising:
treating, at a hydrotreater unit, the pentane hydrocarbon stream to remove impurities prior to supplying the pentane hydrocarbon stream to the first reverse isomerization unit;
cracking the n-pentane rich stream to produce a mixed ethylene stream containing ethylene, one or more additional cracked products, n-pentane and iso-pentane;
separating, at one or more cracker separation units, the mixed ethylene stream to produce an ethylene stream and a recycled pentane stream containing n-pentane and iso-pentane;
combining the recycled pentane stream and an iso-pentane stream derived from N GLs to form the pentane hydrocarbon stream; and
hydrotreating the iso-pentane stream derived from NGLs at a hydrotreater to form a hydrotreated iso-pentane stream, and combining the hydrotreated iso-pentane stream with the recycled pentane stream before being fed to the first reverse isomerization unit.

8. The method according claim 1, wherein the second isomerized pentane stream contains n-pentane, iso-pentane, and neo-pentane, and wherein prior to combining the second isomerized pentane stream and the n-pentane stream, and the method further comprises:
separating, at a neo-pentane separation unit, neo-pentane from the second isomerized pentane stream to produce a neo-pentane stream and an enriched second isomerized pentane stream containing n-pentane and iso-pentane;
combining the enriched second isomerized pentane stream and the first n-pentane stream to produce the n-pentane rich stream; and
feeding the neo-pentane stream to the first reverse isomerization unit.

9. The method according to claim 1, wherein prior to combining the second isomerized pentane stream and the n-pentane stream, the method further comprises:
separating, at a second separation unit, n-pentane from the second isomerized pentane stream to generate a second n-pentane stream and a second iso-pentane rich stream;
supplying the second iso-pentane rich stream to a third reverse isomerization unit to produce a third isomerized pentane stream containing n-pentane, iso-pentane, and neo-pentane;
combining the third isomerized pentane stream and the first and second n-pentane streams to produce the n-pentane rich stream.

10. The method according to claim 1, wherein prior to combining the second isomerized pentane stream and the n-pentane stream, the method further comprises:
separating, at a second separation unit, n-pentane from the second isomerized pentane stream to generate the second n-pentane stream and the second iso-pentane rich stream;
supplying the second iso-pentane rich stream to a third reverse isomerization unit to produce the third isomerized pentane stream containing n-pentane, iso-pentane, and neo-pentane;
separating, at a neo-pentane separation unit, neo-pentane from the third isomerized pentane stream to produce a neo-pentane stream and an enriched third isomerized pentane stream containing n-pentane and iso-pentane;
combining the enriched third isomerized pentane stream and the first and the second n-pentane streams to produce the n-pentane rich stream.

11. The method according to claim 9, wherein the first separation unit and the second separation unit are one or more of a distillation column or a crystallization unit; wherein at least one of the first reverse isomerization unit, the second reverse isomerization unit, and the third reverse isomerization unit is configured to operate in the presence of a platinum or a palladium catalyst supported on a solid matrix; wherein the solid matrix is a chlorinated alumina, a zeolite matrix, a mordenite matrix, a sulfated zirconia, or any combination thereof; and wherein at least one of the first reverse isomerization unit, the second reverse isomerization unit, and the third reverse isomerization unit is configured to operate at a temperature of from about 150° C. to about 450° C. in the presence of hydrogen.

12. A system for the production of ethylene from a pentane hydrocarbon stream containing iso-pentane, the system comprising:
a first reverse isomerization unit operable to receive a pentane stream and allow for isomerization of the pentane stream to produce a first isomerized pentane stream containing n-pentane and iso-pentane;
a first separation unit operable to receive the first isomerized pentane stream and allow for separation of n-pentane from the isomerized pentane stream to form a first n-pentane stream and an iso-pentane rich stream;
a second reverse isomerization unit operable to receive the iso-pentane rich stream and allow for isomerization of the iso-pentane rich stream to produce a second isomerized pentane stream containing n-pentane and iso-pentane; and
a steam cracker operable to receive the second isomerized pentane stream and the first n-pentane stream as a n-pentane rich stream, the steam cracker further operable to crack the n-pentane rich stream to produce a cracked product containing ethylene and propylene.

13. The system according to claim 12, further comprising:
a hydrotreater unit operable to receive the pentane hydrocarbon stream and remove impurities from the pentane hydrocarbon stream to form a purified pentane stream, the hydrotreater unit further operable to feed the purified pentane stream to the first reverse isomerization unit;
one or more cracker units to crack the n-pentane rich stream to produce a mixed ethylene stream containing ethylene, pentane, and one or more additional cracked products; and
one or more cracker separation units operable to receive the mixed ethylene stream from the steam cracker and to produce an ethylene stream and a recycled pentane stream;
wherein the first reverse isomerization unit is operable to receive a combined pentane stream containing the recycled pentane stream and the pentane stream or the purified pentane stream.

14. The system according to claim 12, wherein the second isomerized pentane stream contains n-pentane, iso-pentane, and neo-pentane, and wherein the system further comprises:
a neo-pentane separation unit operable to receive the second isomerized pentane stream and separate neo-pentane from the second isomerized pentane stream to produce a neo-pentane stream and an enriched second isomerized pentane stream containing n-pentane and iso-pentane, the steam cracker operable to receive the combined n-pentane rich stream containing the combination of the first n-pentane stream and the enriched second isomerized pentane stream, the steam cracker further operable to crack the n-pentane rich stream to produce ethylene and one or more additional cracked products;
a second separation unit operable to receive the second isomerized pentane stream and separate n-pentane from the second isomerized pentane stream to generate a second n-pentane stream and a second iso-pentane rich stream; and
a third reverse isomerization unit operable to receive the second iso-pentane rich stream and isomerize the second iso-pentane rich stream to produce a third isomerized pentane stream containing n-pentane, iso-pentane, and neo-pentane;
wherein the steam cracker is operable to receive a combined n-pentane rich stream containing the combination of the first n-pentane stream, the second n-pentane stream, and the third isomerized pentane stream, the steam cracker further operable to crack the n-pentane rich stream to produce ethylene and one or more additional cracked products.

15. The system according to claim 14, wherein at least one of the first reverse isomerization unit, the second reverse isomerization unit, and the third reverse isomerization unit is configured to operate in the presence of a platinum or a palladium catalyst supported on a solid matrix; wherein the solid matrix is a chlorinated alumina, a zeolite matrix, a mordenite matrix, a sulfated zirconia, or any combination thereof; and wherein at least one of the first reverse isomerization unit, the second reverse isomerization unit, and the third reverse isomerization unit is configured to operate at a temperature of from about 150° C. to about 450° C. in the presence of hydrogen.
